(19) United States
(12) Patent Application Publication
(10) Pub. No.: US 2018/0318214 A1
(43) Pub. Date: Nov. 8, 2018

(54) PHARMACEUTICAL COMPOSITIONS OF DRONABINOL

(71) Applicant: LUPIN ATLANTIS HOLDINGS SA, Zug (CH)

(72) Inventors: Anandhavalavan Arulmozhi, Somerset, NJ (US); James Garegnani, Somerset, NJ (US)

(73) Assignee: LUPIN ATLANTIS HOLDINGS SA, Zug (CH)

(21) Appl. No.: 15/969,287

(22) Filed: May 2, 2018

Related U.S. Application Data
(60) Provisional application No. 62/500,246, filed on May 2, 2017.

(51) Int. Cl. A61K 9/08; A61K 9/00; A61K 31/353; A61K 47/10; A61K 47/22

(52) U.S. Cl. CPC A61K 9/08; A61K 9/0053; A61K 47/22; A61K 47/10; A61K 31/353

(57) ABSTRACT

The present invention relates to pharmaceutical compositions of dronabinol and process for preparation thereof. The stable oral solution of dronabinol can be formulated by further reducing the amount of water to less than about 25% w/w of the composition. The solution of the invention was found to be stable at various temperature and humidity conditions for prolonged periods of time. The invention relates to dronabinol compositions administered through oral route in the form of solutions.

### TECHNICAL FIELD OF THE INVENTION

The present invention relates to pharmaceutical compositions of dronabinol and process for preparation thereof. More preferably, the invention relates to dronabinol compositions administered through oral route in the form of solutions.

### BACKGROUND OF THE INVENTION

Delta-9-Tetrahydrocannabinol (also known as THC, dronabinol and D9THC) is a naturally occurring compound and is the primary active ingredient in the controlled substance marijuana. Marijuana refers to the dried flowers and leaves of Cannabis Sativa, the hemp plant. These parts of the plant contain several compounds called cannabinoids (including dronabinol), that may help patients with certain disease conditions. Dronabinol has been approved by the Food and Drug Administration (FDA) for the control of nausea and vomiting associated with chemotherapy and, more recently, for appetite stimulation of AIDS patients suffering from wasting syndrome. Synthetic dronabinol has been utilized as a pharmaceutically active ingredient, and cannabis-based medicines using botanical sources of cannabis rather than synthetic THC are also known in the art.

Dronabinol is a cannabinoid designated chemically as (6aR,10aR)-6a,7,8,10a-Tetrahydro-6,6,9-trimethyl-3pentyl-6H-dibenzo[b,d]-pyran-1-ol. Dronabinol has the following structural formula:

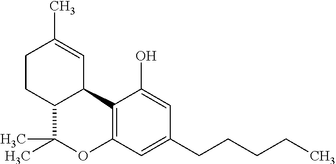 [SMILES: CCCCCc1cc(O)c2c(c1)OC(C)(C)[C@@H]1CCC(C)=C[C@@H]21]

Numerous work has been carried out in the prior art on the cannabinoids. U.S. Pat. No. 8,222,292 discloses oral solutions of dronabinol wherein the amount of water in the solution is 30-33% of the composition. U.S. Pat. No. 9,345,771 discloses oral solutions of dronabinol wherein the amount of water in the solution is 25-33% of the composition. It further discloses the use of a cosolvent consisting of 5.5% w/w propylene glycol and 12% w/w polyethylene glycol of the composition. The applicant claims stable solution at room temperature or refrigerated temperature which may have improved in vivo absorption profiles with faster onset and lower inter-subject variability.

### DESCRIPTION OF THE INVENTION

The inventors of the present invention found that the stable oral solution of dronabinol can be formulated by further reducing the amount of water to less than about 25% w/w of the composition. Further, the inventors formulated solutions with and amount of water more than about 33%, which was surprisingly found to be stable on stability.

The solution of the invention was found to be stable at various temperature and humidity conditions for prolonged periods of time without any issue. The solution of dronabinol of the invention may possess improved in vivo absorption profiles with faster onset and lower inter-subject variability. The dronabinol solution of the invention may be bioequivalent to a marketed composition of dronabinol.

It is one object of the invention to provide oral pharmaceutical compositions comprising dronabinol.

In one embodiment, oral pharmaceutical compositions comprise 0.1 to 5% w/w of dronabinol, water, solvent, and cosolvent selected from the group consisting of propylene glycol, polyethylene glycol, glycerin and combinations thereof, and optionally comprising one or more pharmaceutically acceptable excipients, and having a combined total of 100%, wherein the amount of water in the solution is more than about 33% w/w.

In preferred embodiment, the amount of water in the solution is more than 34% w/w, 35% w/w, 38% w/w or 40% w/w.

In one embodiment, the oral pharmaceutical composition comprises alcohol as the solvent. In a preferred embodiment the alcohol is ethanol.

In another embodiment, the oral pharmaceutical composition contains about 50% of alcohol.

In all embodiments of the invention, the combined total of all ingredients is 100%.

All percentages of ingredients reported herein are expressed as weight by weight, unless otherwise indicated.

It is an object of the invention to provide methods of treatment wherein the oral dronabinol compositions are preferably in the form of solutions that are suitable for administration by the oral route.

In another embodiment, oral pharmaceutical compositions of the invention are stable under multiple conditions, including refrigerated, cool and room temperature storage conditions. In more preferred embodiment, the stabilized pharmaceutical compositions, preferably in the form of solutions, may be stored at ambient temperature and humidity, or in a refrigerator.

In another embodiment, oral pharmaceutical compositions of the invention comprise effective amounts of one or more stabilizers to promote stability of the cannabinoid against unacceptable degradation. The stabilizers may comprise one or more anti-oxidants, one or more organic bases, and/or other stabilizers for cannabinoids known to those skilled in the art. In certain preferred embodiments, the stabilizer comprises butylated hydroxyanisole (BHA) or butylated hydroxytoluene (BHT) or any combination thereof.

The invention is further directed in part to a method for stabilizing pharmaceutical compositions containing dronabinol as the active pharmaceutical ingredient, comprising dissolving a therapeutically effective amount of dronabinol in a mixture of aqueous and organic carriers. In certain embodiments, the carrier comprises buffering agents. In yet another embodiment, the carrier further comprises one or more stabilizers for the dronabinol (e.g, anti-oxidants, organic bases, or both, as set forth more specifically herein).

In another embodiment, the carrier further contains an effective amount of a viscosity modifier that may be included to provide a pharmaceutically acceptable viscosity to the dronabinol dispersed in the carrier.

In another embodiment where the stabilizer comprises an organic base, the dosage form may comprise an organic base. In another embodiment, the organic base is selected from the group consisting of butylated hydroxyanisole (BHA), butylated hydroxytoluene (BHT), ascorbic acid, sodium ascorbate, ascorbyl palmitate and any combination of the foregoing.

In one embodiment, the oral pharmaceutical compositions may comprise 0.1 to 5% w/w of dronabinol, water, solvent, and cosolvent selected from the group consisting of propylene glycol, polyethylene glycol, glycerin and combinations thereof, optionally comprising one or more pharmaceutically acceptable excipients, having a combined total of 100%, wherein the amount of water in the solution is more than about 33% w/w, and wherein the composition is devoid of butylated hydroxyanisole (BHA).

The anti-oxidant included in the pharmaceutical composition of the invention may further be selected from, e.g., propyl gallate, lecithin, Vitamin E tocopherol, sesamin, sesamol, sesamolin, alpha tocopherol, ascorbic acid, ascorbyl palmitate, fumaric acid, malic acid, and sodium metabisulphite, disodium ethylenediaminetetracetic acid (EDTA), and combinations of any of the foregoing.

In one embodiment, the oral pharmaceutical composition comprises 0.1 to 5% w/w of dronabinol, water, solvent, and cosolvent selected from the group consisting of propylene glycol, polyethylene glycol, glycerin and combinations thereof, optionally comprising one or more pharmaceutically acceptable excipients, having a combined total of 100%, wherein the amount of water in the solution is more than about 33% w/w, and wherein the composition is devoid of disodium ethylenediaminetetraacetic acid (EDTA).

The oral pharmaceutical compositions of the present invention comprise a dronabinol concentration range of from about 0.01 to about 20 mg/ml. In certain embodiments, the oral pharmaceutical compositions of the invention comprise dronabinol in a concentration of from about 1 to about 10 mg/ml. In yet another embodiment, the oral pharmaceutical compositions comprise dronabinol in a formulation at about 5 mg/ml.

It is to be understood for the purposes of the invention that the term “pharmaceutical composition” or “oral pharmaceutical composition” can be used interchangeably.

In preferred embodiments, the oral pharmaceutical compositions comprise from about 0.05% to about 90% dronabinol, preferably from about 0.1% to about 50% dronabinol, more preferably about 0.1% to about 10% w/w dronabinol, and most preferably from about 0.1% to about 5% dronabinol, by weight.

It is a further object of the invention to provide stabilized pharmaceutical compositions of dronabinol, preferably in the form of solutions where the carrier is buffered to a pH of from about 5 to about 10. In certain other embodiments, the carrier is buffered to a pH of from about 6 to about 8. In accordance with any of the above objects, the solutions of the invention are preferably buffered to a pH of from about 6.5 to about 7.5.

In another embodiment, the pharmaceutical compositions in accordance with any of the above objects may also include sweeteners such as xylitol from about 5% to about 25%; saccharin from about 0.01% to about 5%; and saccharin sodium from about 0.01% to about 5% by weight of the formulation.

The invention is further directed to pharmaceutical compositions which further comprise one or more additional therapeutically active agents. Non-limiting examples of such additional therapeutically active agents include a narcotic analgesic, a non-narcotic analgesic, an anti-emetic, a steroid, and mixtures of any of the foregoing.

In another embodiment, pharmaceutical compositions of the invention include further pharmaceutically acceptable excipients. Non-limiting examples of such pharmaceutically acceptable excipients include solubilizers for said cannabinoid, emulsifiers, absorption enhancers, surfactants, propellants, etc.

The term “pharmaceutically acceptable” is defined for purposes of the invention as meaning that a particular ingredient (e.g., pharmaceutical carrier, excipient) is not biologically or otherwise undesirable in an oral dosage form, i.e., the amount of the compound in an orally administered composition or dosage form does not cause any undesirable effects to the formulation or to the patient.

In another embodiment, the solvents are organic solvents such as dehydrated alcohol, ethanol, propanol, isopropanol, propylene glycol, polyethylene glycol, and combinations thereof that are pharmaceutically acceptable based on the desired formulation. For purposes of this invention, the term “dehydrated alcohol” is used interchangeably with the term “absolute alcohol.” The amount of dehydrated alcohol in a particular formulation will vary based on the intended formulation and the solubility of the dronabinol.

The amount of dehydrated alcohol in the pharmaceutical compositions of the present invention can range from about 10% to about 90%; from about 15% to about 65%; and about 15% to about 50% by weight.

In another embodiment, cosolvents are selected from propylene glycol, polyethylene glycol, glycerin or the like and combinations thereof.

In another embodiment, compositions of the invention further comprise solubilizing agents. Exemplary solubilizing agents include Capryol 90; Cremophor RH40; Labrafil M 1944 CS; Labrafil M 2125 CS; Lauroglycol 90; PEG MW>4000; Plurol Oleique CC 497; poloxamer 124; poloxamer 188; Softigen 701; Softigen 767; Tagat TO; Tween 80; triacetin; triethylcitrate; tributylcitrate; acetyl triethylcitrate; acetyl tributyl citrate; ethyl oleate; ethyl caprylate; ethyl butyrate; triacetin; 2-pyrrolidone; 2-piperidone; N-methylpyrrolidone; N-ethylpyrrolidone; N-hydroxyethyl pyrrolidone; N-octylpyrrolidone; N-laurylpyrrolidone; dimethylacetamide; Miglyol, lanolin, petrolatum, mineral oil and mixtures thereof.

Other components such as preservatives, antioxidants, surfactants, absorption enhancers, viscosity modifiers, bulking agents, diluents, coloring agents, flavoring agents, pH modifiers, sweeteners or taste-masking agents may also be incorporated into any of the pharmaceutical compositions described as part of the invention. The amount of each of these components which may be used will be optimized for each formulation in order to obtain a stable composition, and preferably a solution having the desired shelf-life.

In another embodiment, pharmaceutical compositions of the invention may further comprise amounts of one or more pharmaceutically acceptable anti-oxidants in an amount effective to stabilize the dronabinol contained therein such that the dronabinol does not degrade to an unacceptable extent and the composition is deemed stable for a prolonged period when placed under storage conditions selected from refrigerated or room temperature.

The term “anti-oxidant” is used herein to describe any compound or combination of compounds that prevents or slows down dronabinol oxidation. Any of the known anti-oxidants may be used, including but not limited to anti-oxidants such as butylated hydroxyanisole (BHA), butylated hydroxytoluene (BHT), propyl gallate, lecithin, Vitamin E tocopherol, sesamin, sesamol, sesamolin, alpha tocopherol, ascorbic acid, ascorbyl palmitate, fumaric acid, malic acid, sodium ascorbate and sodium metabisulphite, as well as chelating agents such as disodium EDTA, may also be used to stabilize the dronabinol compositions of the present invention. The composition of invention may also contain anti-oxidant synergists to prevent oxidative degradation. Any of the known anti-oxidant synergists may also be used in effective amounts, for example disodium edetate.

In another embodiment, effective amounts of one or more pharmaceutically acceptable organic or inorganic bases are added to the compositions in order to stabilize the dronabinol from undesirable levels of degradation. Examples of suitable organic bases which may be effectively used in the compositions of the present invention include but are not limited to any pharmaceutically acceptable primary, secondary and tertiary organic amines which are GRAS ingredients (generally regarded as safe), such as methanolamine, ethanolamine, meglumine, other alkylamines (e.g. di-alkyl amines and tri-alkyl amines), and any combination thereof.

In another embodiment, the compositions of the invention further comprise preservatives having bactericidal and fungicidal properties. Parabens are widely used preservatives in the pharmaceutical industry because of their efficacy as preservatives in combination with their long history of safe use. Parabens work best when they are used in combination since various combinations of parabens allow for the use of lower levels while increasing preservative activity. Examples of parabens include methyl paraben and propyl paraben.

In addition, the compostions may additionally include physiologically acceptable components such as sodium chloride and like materials conventionally used to achieve isotonicity with typical body fluids.

Agents which buffer the pH to maintain a physiologically compatible pH range to enhance the solubility and stability of the active agent present, and the like, may also be included in certain embodiments of the present invention. Suitable buffers include, but are not limited to acetate, bicarbonate, citrate, phosphate, pharmaceutically acceptable salts thereof and combinations or mixtures thereof. When one or more buffers are utilized in the formulations of the invention, they may be combined. The concentration of buffer is such that a pH of the formulation is from about 5 to about 10; preferably from about 6 to about 8; more preferably from about 6.5 to about 7.5 and most preferably about 7.

In another embodiment, the pharmaceutical compostions for oral administration may comprise sucrose, fructose, sucralose, sorbitol, xylitol, saccharin, saccharin sodium, or combinations thereof as a sweetening agent.

In further embodiments, the pharmaceutical compositions of invention further comprise one or more viscosity modifiers including, for example, cellulose or cellulose derivatives such as ethylcellulose, hydroxyethylcellulose, hydroxypropylcellulose, carboxymethylcellulose, methylcellulose, sodium hydroxypropylmethylcellulose, methylethylcellulose, sodium carboxymethylcellulose, Aerosil (silicon dioxide), cetostearyl alcohol, cetyl alcohol, stearyl alcohol, Gelucires 33/01, 39/01 and 43/01, glyceryl behenate (Compritol 888 A TO), glyceryl palmitostearate (Precirol AT05), Softisans 100, 142, 378 and 649, stearyl alcohol carbomer, xanthan gum, maltodextrin, acacia, tragacanth, povidone and polyvinyl alcohol.

In another embodiment, the pharmaceutical compostions of inventions further comprise one or more absorption enhancers for use in accordance with certain embodiments of the present invention include, for example, Gelucire 44/14; Gelucire 50/13; Tagat TO; Tween 80; isopropyl myristate, polysorbates, sorbitan esters, poloxamer block copolymers, PEG-35 castor oil, PEG-40 hydrogenated castor oil, caprylocaproyl macrogol-8 glycerides, PEG-8 caprylic/capric glycerides, sodium lauryl sulfate, dioctyl sulfosuccinate, polyethylene lauryl ether, ethoxydiglycol, propylene glycol mono-di-caprylate, glycerol monocaprylate, glyceryl fatty acids (C8-C18) ethoxylated, oleic acid, linoleic acid, glyceryl caprylate/caprate, glyceryl monooleate, glyceryl monolaurate, caprylic/capric triglycerides, ethoxylated nonylphenols, PEG-(8-50) stearates, olive oil PEG-6 esters, triolein PEG-6 esters, lecithin, d-alpha tocopheryl polyethylene glycol 1000 succinate, polycarbonate, sodium glycocholate, sodium taurocholate, cyclodextrins, citric acid, sodium citrate, triacetin, combinations thereof, and the like.

In another embodiment, the pharmaceutical compositions of the present invention also may contain one or more suitable gelling or suspension agents include carbomers, modified cellulose derivatives, naturally-occurring, synthetic or semi-synthetic gums such as xanthan gum, acacia and tragacanth, modified starches, co-polymers such as those formed between maleic anhydride and methyl vinyl ether, colloidal silica and methacrylate derivatives or a mixture thereof.

In further embodiments, additional excipients compatible with the pharmaceutical compositions of the invention may be incorporated into the compostions, if needed, such as known surfactants (e.g. Capryol 90; Cremophor RH40; Gelucire 44/14; Gelucire 50/13; Imwitor 91; Imwitor 308; Imwitor 380; Imwitor 742; Imwitor 780K; Imwitor 928; Imwitor 988; Labrafil M 1944 CS; Labrafil M 2125 CS; Lauroglycol 90; Tagat TO; Tween 80; and mixtures thereof); and emulsifiers (e.g., Gelucire 44/14; Gelucire 50/13; Imwitor 91; Imwitor 308; Imwitor 380; Imwitor 742; Imwitor 780K; Imwitor 928; Imwitor 988; poloxamer 124; poloxamer 188; Tagat TO; Tween 80; lecithin; lysolecithin; phosphatidylcholine; phosphatidylethanolamine; phosphatidylglycerol; phosphatidic acid; phosphatidylserine; lysophosphatidylcholine; lysophosphatidylethanolamine; lysophosphatidylglycerol; lysophosphatidic acid; lysophosphatidylserine; PEG-phosphatidylethanolamine; PVP-phosphatidylethanolamine; sodium lauryl sulfate and mixtures thereof).

Other additives conventionally used in the pharmaceutical compositions can be included, and these additives are well known in the art. Such additives include pharmaceutically acceptable detackifiers, anti-foaming agents, chelating agents, viscomodulators, tonicifiers, flavorants, colorants odorants, opacifiers, suspending agents, binders, fillers, plasticizers, lubricants, and mixtures thereof.

The amounts of such additives can be readily determined by one skilled in the art according to the particular properties desired, keeping in mind the possibility that any such additives should preferably not negatively impact the stability of the final formulation.

Suitable coloring agents include red, black and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable sweeteners include sucralose, xylitol, saccharin, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ion-exchange resins, cyclodextrin inclusion compounds, adsorbates, and the like.

It is recognized that pharmaceutical excipients may perform more than one function, and are therefore characterized as having different uses depending on the particular application. While the use of an excipient in the context of a particular formulation may determine the function of the excipient, the inclusion of any particular excipient into any one or more categories as set forth above is not meant to limit the function of that excipient.

In another embodiment, oral pharmaceutical compositions comprise 0.1% to 5% w/w of dronabinol, water, about 50% w/w alcohol, cosolvent selected from the group consisting of propylene glycol, glycerin and combinations thereof, optionally comprising one or more pharmaceutically acceptable excipients, having a combined total of 100%, wherein the amount of water in the solution is more than about 33% w/w.

In another embodiment, oral pharmaceutical compositions comprise 0.1% to 5% w/w of dronabinol, water, about 50% w/w alcohol, and cosolvent selected from the group consisting of propylene glycol, glycerin and combinations thereof, optionally comprising one or more pharmaceutically acceptable excipients, having a combined total of 100%, and wherein the composition is devoid of butylated hydroxyanisole (BHA).

In another embodiment, oral pharmaceutical compositions comprise 0.1% to 5% w/w of dronabinol, water, about 50% w/w alcohol, and cosolvent selected from the group consisting of propylene glycol, glycerin and combinations thereof, optionally comprising one or more pharmaceutically acceptable excipients, having a combined total of 100%, and wherein the composition is devoid of disodium ethylenediaminetetracetic acid (EDTA).

In another embodiment, oral pharmaceutical compositions comprise 0.1% to 5% w/w of dronabinol, water, about 50% w/w ethanol, and cosolvent selected from the group consisting of propylene glycol, glycerin and combinations thereof, optionally comprising one or more pharmaceutically acceptable excipients, having a combined total of 100%, wherein the amount of water in the solution is more than about 33% w/w.

In another embodiment, oral pharmaceutical compositions comprise 0.1% to 5% w/w of dronabinol, water, about 50% w/w alcohol, and cosolvent, and optionally comprising one or more pharmaceutically acceptable excipients selected from propyl gallate, ethyl gallate, Vitamin E polyethylene glycol succinate, propyl paraben or methyl paraben, having a combined total of 100%, wherein the cosolvent consists of 5.5% w/w propylene glycol and 5% w/w glycerin.

In another embodiment, oral pharmaceutical compositions comprise 0.1% to 5% w/w of dronabinol, water, about 50% w/w alcohol, and cosolvent, and optionally comprising one or more pharmaceutically acceptable excipients, having a combined total of 100%, wherein the cosolvent consists of 5.5% w/w propylene glycol and 6.5% w/w polyethylene glycol.

In another embodiment, oral pharmaceutical compositions comprise 0.1% to 5% w/w of dronabinol, water, about 50% w/w alcohol, cosolvent, and optionally comprising one or more pharmaceutically acceptable excipients selected from propyl gallate, ethyl gallate, Vitamin E polyethylene glycol succinate, propyl paraben or methyl paraben, having a combined total of 100%, wherein the cosolvent consists of 5.5% w/w propylene glycol and 6.5% w/w polyethylene glycol.

In another embodiment, oral pharmaceutical compositions comprise 0.1% to 5% w/w of dronabinol, water, about 50% w/w alcohol, and cosolvent selected from the group consisting of propylene glycol, polyethylene glycol, glycerin and combinations thereof, and optionally comprising one or more pharmaceutically acceptable excipients, having a combined total of 100%, wherein the amount of water in the solution is less than about 25% w/w.

In another embodiment, oral pharmaceutical compositions comprise 0.1% to 5% w/w of dronabinol, water, about 50% w/w alcohol, and cosolvent, and optionally comprising one or more pharmaceutically acceptable excipients, having a combined total of 100%, wherein the cosolvent consists of 5.5% w/w propylene glycol and 18.5% w/w polyethylene glycol.

In another embodiment, oral pharmaceutical compositions comprise 0.1% to 5% w/w of dronabinol, water, about 50% w/w alcohol, and cosolvent, and optionally comprising one or more pharmaceutically acceptable excipients selected from propyl gallate, ethyl gallate, Vitamin E polyethylene glycol succinate, propyl paraben or methyl paraben, and having a combined total of 100%, wherein the cosolvent consists of 5.5% w/w propylene glycol and 18.5% w/w polyethylene glycol.

In another embodiment, oral pharmaceutical compositions comprise 0.1% to 5% w/w of dronabinol, water, about 50% w/w alcohol, and cosolvent, and optionally comprising one or more pharmaceutically acceptable excipients selected from propyl gallate, ethyl gallate, Vitamin E polyethylene glycol succinate, propyl paraben or methyl paraben, having a combined total of 100%, wherein the cosolvent consists of 5.5% w/w propylene glycol, 18.5% w/w polyethylene glycol and 5% w/w glycerin.

In another embodiment, oral pharmaceutical compositions comprise 0.1% to 5% w/w of dronabinol, water, about 50% w/w alcohol, and cosolvent, and optionally comprising one or more pharmaceutically acceptable excipients selected from propyl gallate, ethyl gallate, Vitamin E polyethylene glycol succinate, propyl paraben or methyl paraben, having a combined total of 100%, wherein the cosolvent consisting of 5.5% w/w propylene glycol and 5% w/w glycerin.

Many other variations of the present invention will be apparent to those skilled in the art and are meant to be within the scope of the claims appended hereto. The foregoing specification alludes to beliefs, hypothesis and conclusions of the inventors based on their experience in the field, the reports of others and experiments conducted and reported herein, and are provided for purposes of (possible) explanation only and are not meant to limit the invention in any manner whatsoever.

The following examples illustrate various aspects of the present invention, and are set forth to assist in understanding the invention. These examples should not be construed as specifically limiting the invention described and claimed herein. Variations of the invention, including the substitution of all equivalents now known or later developed, which would be within the purview of those skilled in the art, and changes in formulation or minor changes in experimental design, are considered to fall within the scope of the invention and appended claims.

### Example 1

| | Quantity |
|---|---|
| Ingredient | (% w/w) |
| Dronabinol | 0.54 |
| Ethyl Alcohol 200 proof | QS |
| Propylene Glycol | 5.50 |
| Glycerin | 5.00 |
| Purified Water, USP | 38.50 |
| Propyl Gallate | 0.01 |
| Ethyl Gallate | 0.01 |
| Propyl paraben | 0.02 |
| Methyl paraben | 0.02 |
| Sucralose | 0.05 |
| Total | 100 |

### Process:

1) An excipient solution is prepared by dissolving Propyl Gallate, Ethyl Gallate, Methyl Paraben and Propyl Paraben in dehydrated Alcohol.
2) Glycerin, Propylene Glycol, Water and Sucralose are then added to the above solution while continuing to mix in the container.
3) The contents are then purged with nitrogen or alternatively an air tight container with nitrogen purging capability can be used for preparation of the excipient solution.
4) Then, a calculated amount of dronabinol in ethanol solution is added to the excipient solution and mixed for about fifteen minutes to ninety minutes preferably for thirty to forty five minutes while continuing to be purged with nitrogen in an airtight container.
5) The volume is adjusted to a required amount by adding dehydrated alcohol, then mixed for about ten minutes while the mixture continues to be purged with nitrogen.
6) The dronabinol solution is then filtered and filled into 30 ml amber glass bottles under a nitrogen gas blanketed atmosphere.

### Example 2

| | Quantity |
|---|---|
| Ingredient | (% w/w) |
| Dronabinol | 0.54 |
| Ethyl Alcohol 200 proof | QS |
| Propylene Glycol | 5.50 |
| PEG 400 | 18.50 |
| Glycerin | 5.00 |
| Purified Water, USP | 20.00 |
| Propyl Gallate | 0.01 |
| Ethyl Gallate | 0.01 |
| Propyl paraben | 0.02 |
| Methyl paraben | 0.02 |
| Sucralose | 0.05 |
| Total | 100 |

### Process:

1) An excipient solution is prepared by dissolving Propyl Gallate, Ethyl Gallate, Methyl Paraben and Propyl Paraben in dehydrated Alcohol.
2) PEG 400, Glycerin, Propylene Glycol, Water and Sucralose are then added to the above solution while continuing to mix in the container.
3) The contents are then purged with nitrogen or alternatively an air tight container with nitrogen purging capability can be used for preparation of the excipient solution.
4) Then, a calculated amount of dronabinol in ethanol solution is added to the excipient solution and mixed for about fifteen minutes to ninety minutes preferably for thirty to forty five minutes while continuing to be purged with nitrogen in an airtight container.
5) The volume is adjusted to a required amount by adding dehydrated alcohol, then mixed for about ten minutes while the mixture continues to be purged with nitrogen.
6) The dronabinol solution is then filtered and filled into 30 ml amber glass bottles under a nitrogen gas blanketed atmosphere.

### Example 3

| | Quantity |
|---|---|
| Ingredient | (% w/w) |
| Dronabinol | 0.54 |
| Ethyl Alcohol 200 proof | QS |
| Propylene Glycol | 5.50 |
| PEG 400 | 6.50 |
| Purified Water, USP | 37.00 |
| Propyl Gallate | 0.01 |
| Ethyl Gallate | 0.01 |
| Propyl paraben | 0.02 |
| Methyl paraben | 0.02 |
| Sucralose | 0.05 |
| Total | 100 |

### Process:

1) An excipient solution is prepared by dissolving Propyl Gallate, Ethyl Gallate, Methyl Paraben and Propyl Paraben in dehydrated Alcohol.
2) PEG 400, Glycerin, Propylene Glycol, Water and Sucralose are then added to the above solution while continuing to mix in the container.
3) The contents are then purged with nitrogen or alternatively an air tight container with nitrogen purging capability can be used for preparation of the excipient solution.
4) Then, a calculated amount of dronabinol in ethanol solution is added to the excipient solution and mixed for about fifteen minutes to ninety minutes preferably for thirty to forty five minutes while continuing to be purged with nitrogen in an airtight container.
5) The volume is adjusted to a required amount by adding dehydrated alcohol, then mixed for about ten minutes while the mixture continues to be purged with nitrogen.
6) The dronabinol solution is then filtered and filled into 30 ml amber glass bottles under a nitrogen gas blanketed atmosphere.

### Example 4

| | Quantity |
|---|---|
| Ingredient | (% w/w) |
| Dronabinol | 0.54 |
| Ethyl Alcohol 200 proof | QS |
| Propylene Glycol | 5.50 |
| PEG 400 | 18.50 |
| Glycerin | 5.00 |
| Purified Water, USP | 20.00 |
| Vitamin E Polyethylene | 0.01 |
| Glycol Succinate | |
| Propyl paraben | 0.02 |
| Methyl paraben | 0.02 |
| Sucralose | 0.05 |
| Total | 100 |

### Process:

1) An excipient solution is prepared by dissolving Methyl Paraben and Propyl Paraben in dehydrated Alcohol.
2) PEG 400, Glycerin, Propylene Glycol, Vitamin E Polyethylene Glycol Succinate, Water and Sucralose are then added to the above solution while continuing to mix in the container.
3) The contents are then purged with nitrogen or alternatively an air tight container with nitrogen purging capability can be used for preparation of the excipient solution.
4) Then, a calculated amount of dronabinol in ethanol solution is added to the excipient solution and mixed for about fifteen minutes to ninety minutes preferably for thirty to forty five minutes while continuing to be purged with nitrogen in an airtight container.
5) The volume is adjusted to a required amount by adding dehydrated alcohol, then mixed for about ten minutes while the mixture continues to be purged with nitrogen.
6) The dronabinol solution is then filtered and filled into 30 ml amber glass bottles under a nitrogen gas blanketed atmosphere.

### Example 5

| | Quantity |
|---|---|
| Ingredient | (% w/w) |
| Dronabinol | 0.54 |
| Ethyl Alcohol 200 proof | QS |
| Propylene Glycol | 5.50 |
| Glycerin | 5.00 |
| Purified Water, USP | 38.50 |
| Vitamin E Polyethylene | 0.01 |
| Glycol Succinate | |
| Propyl paraben | 0.02 |
| Methyl paraben | 0.02 |
| Sucralose | 0.05 |
| Total | 100 |

### Process:

1) An excipient solution is prepared by dissolving Methyl Paraben and Propyl Paraben in dehydrated Alcohol.
2) Glycerin, Propylene Glycol, Vitamin E Polyethylene Glycol Succinate, Water and Sucralose are then added to the above solution while continuing to mix in the container.
3) The contents are then purged with nitrogen or alternatively an air tight container with nitrogen purging capability can be used for preparation of the excipient solution.
4) Then, a calculated amount of dronabinol in ethanol solution is added to the excipient solution and mixed for about fifteen minutes to ninety minutes preferably for thirty to forty five minutes while continuing to be purged with nitrogen in an airtight container.
5) The volume is adjusted to a required amount by adding dehydrated alcohol, then mixed for about ten minutes while the mixture continues to be purged with nitrogen.
6) The dronabinol solution is then filtered and filled into 30 ml amber glass bottles under a nitrogen gas blanketed atmosphere.

### Example 6

| | Quantity |
|---|---|
| Ingredient | (% w/w) |
| Dronabinol | 0.54 |
| Ethyl Alcohol 200 proof | QS |
| Propylene Glycol | 5.50 |
| PEG 400 | 6.50 |
| Purified Water, USP | 37.00 |
| Vitamin E Polyethylene | 0.01 |
| Glycol Succinate | |
| Propyl paraben | 0.02 |
| Methyl paraben | 0.02 |
| Sucralose | 0.05 |
| Total | 100 |

### Process:

1) An excipient solution is prepared by dissolving Methyl Paraben and Propyl Paraben in dehydrated Alcohol.
2) PEG 400, Glycerin, Propylene Glycol, Vitamin E Polyethylene Glycol Succinate, Water and Sucralose are then added to the above solution while continuing to mix in the container.
3) The contents are then purged with nitrogen or alternatively an air tight container with nitrogen purging capability can be used for preparation of the excipient solution.
4) Then, a calculated amount of dronabinol in ethanol solution is added to the excipient solution and mixed for about fifteen minutes to ninety minutes preferably for thirty to forty five minutes while continuing to be purged with nitrogen in an airtight container.
5) The volume is adjusted to a required amount by adding dehydrated alcohol, then mixed for about ten minutes while the mixture continues to be purged with nitrogen.
6) The dronabinol solution is then filtered and filled into 30 ml amber glass bottles under a nitrogen gas blanketed atmosphere.

We claim:

1. An oral pharmaceutical composition comprise 0.1% to 5% w/w of dronabinol, water, about 50% w/w alcohol, cosolvent selected from the group consisting of propylene glycol, glycerin and combinations thereof, optionally comprising one or more pharmaceutically acceptable excipients, wherein the amount of water in the composition is more than about 33% w/w.

2. The oral pharmaceutical composition according to claim 1, comprises 0.54% w/w of dronabinol.

3. The oral pharmaceutical composition according to claim 2, is solution.

4. An oral pharmaceutical compositions comprise 0.1% to 5% w/w of dronabinol, water, about 50% w/w alcohol, and cosolvent selected from the group consisting of propylene glycol, glycerin and combinations thereof, optionally comprising one or more pharmaceutically acceptable excipients, wherein the amount of water in the composition is more than about 33% w/w and said composition is devoid of butylated hydroxyanisole (BHA).

5. The oral pharmaceutical composition according to claim 4, comprises 0.54% w/w of dronabinol.

6. The oral pharmaceutical composition according to claim 5, is solution.

7. An oral pharmaceutical compositions comprise 0.1% to 5% w/w of dronabinol, water, about 50% w/w alcohol, and cosolvent selected from the group consisting of propylene glycol, glycerin and combinations thereof, optionally comprising one or more pharmaceutically acceptable excipients, wherein the amount of water in the composition is more than about 33% w/w and said composition is devoid of butylated hydroxytoluene (BHT).

8. The oral pharmaceutical composition according to claim 7, comprises 0.54% w/w of dronabinol.

9. The oral pharmaceutical composition according to claim 8, is solution.

10. An oral pharmaceutical compositions comprise 0.1% to 5% w/w of dronabinol, water, about 50% w/w alcohol, and cosolvent selected from the group consisting of propylene glycol, glycerin and combinations thereof, optionally comprising one or more pharmaceutically acceptable excipients, wherein the amount of water in the composition is more than about 33% w/w and said composition is devoid of disodium ethylenediaminetetraacetic acid (EDTA).

11. The oral pharmaceutical composition according to claim 10, comprises 0.54% w/w of dronabinol.

12. The oral pharmaceutical composition according to claim 11, is solution.

13. A oral pharmaceutical compositions comprise 0.1% to 5% w/w of dronabinol, water, about 50% w/w alcohol, and cosolvent selected from the group consisting of propylene glycol, polyethylene glycol, glycerin and combinations thereof, and optionally comprising one or more pharmaceutically acceptable excipients wherein the amount of water in the composition is less than about 25%.

14. The oral pharmaceutical composition according to claim 13, comprises 0.54% w/w of dronabinol.

15. The oral pharmaceutical composition according to claim 14, is solution.

16. An oral pharmaceutical compositions comprise 0.1% to 5% w/w of dronabinol, water, about 50% w/w alcohol, cosolvent, and comprising one or more pharmaceutically acceptable excipients selected from propyl gallate, ethyl gallate, Vitamin E polyethylene glycol succinate, propyl paraben or methyl paraben, wherein the cosolvent consists of 5.5% w/w propylene glycol, 18.5% w/w polyethylene glycol and 5% w/w glycerin.

17. The oral pharmaceutical composition according to claim 16, comprises 0.54% w/w of dronabinol.

18. The oral pharmaceutical composition according to claim 17, is solution.
